(19) United States
(12) Patent Application Publication
(10) Pub. No.: US 2005/0272700 A1
(43) Pub. Date: Dec. 8, 2005

(54) Topical treatment and prevention of human papilloma virus (HPV) infection

(76) Inventors: Servet Buyuktimkin, Robbinsville, NJ (US); Nadir Buyuktimkin, Robbinsville, NJ (US); James L. Yeager, Lake Forest, IL (US)

Correspondence Address: WELSH & KATZ, LTD, 120 S RIVERSIDE PLAZA, 22ND FLOOR, CHICAGO, IL, 60606, US

(21) Appl. No.: 11/125,478

(22) Filed: May 9, 2005

Related U.S. Application Data
(60) Provisional application No. 60/569,544, filed on May 10, 2004.

(51) Int. Cl. A61K 31/724; A61K 31/404

(52) U.S. Cl. 514/58; 514/414; 514/415

(57) ABSTRACT

The present invention contemplates a topical pharmaceutical composition that comprises a medium containing an anti-viral-effective amount of the dietary indole or derivative thereof admixed with a dietary indole compound-complexing amount of a hydroxypropyl-β-cyclodextrin dissolved or dispersed therein. Another embodiment utilizes sorbitol to stabilize the indole. The indole compound remains stable to degradation longer in the presence of the cyclodextrin compound that without it. The composition provides a stabilized aqueous indole composition that can be used to topically treat or prevent human papilloma virus (HPV) infection.

### CROSS-REFERENCE TO RELATED APPLICATION

This application claims the benefit of the filing date of U.S. Application Ser. No. 60/569,544, filed May 10, 2004.

### TECHNICAL FIELD

The present invention relates to a stable composition for use as a non-irritating and non-toxic topical treatment or prevention of human papilloma virus (HPV) infection. More particularly, this invention relates to a water-based, chemically stable formulation of a dietary indole with hydroxypropyl-β-cyclodextrin for topical use at the site of HPV infection.

### BACKGROUND OF THE INVENTION

Each year millions of women in the world are diagnosed with cervical dysplasia. The term cervical dysplasia refers to the appearance of abnormal cells on the surface of the cervix. Over time, these abnormal cells can progress to invasive cervical cancer. There is substantial epidemiological and experimental evidence which indicates that human papilloma virus (HPV) is the causative agent in recurrent and persistent cervical dysplasia and, ultimately, cervical cancer.

HPV is a member of the Papova virus group that is comprised of about 80 different genotypes. At the present time, at least 15 HPV types (HPV16, 18, 31, 33, 35, 39, 45, 51, 52, 56, 58, 59, 66 and 68) have been associated with cervical cancer. The International Agency for Research on Cancer has recently classified high-risk types 16 and 18 as definite carcinogens in humans [IARC Monographs on the evaluation of carcinogenic risks to humans, 64, Lyon, (1995)], with HPV 16 recognized as the most prevalent high risk HPV type [Walboomers et al., J. Pathol. 189:12-19 (1999); Kirwan and Herrington, Br. J. Ostet. Gynaecol. 108:1204-1213 (2001)].

Besides causing cervical changes, HPV infections can also cause various warts, anal papilloma, vaginal epithelial dysplasia, oral infections such as recurrent laryngeal papillomatosis, and oropharyngeal human papilloma virus related papillomatosis with dysplasia. It is estimated that respiratory related papillomas can affect about 15,000 people in the United States, with around 6000 of those being children. Diagnosis and treatment costs over $151 million annually [Derkay, Archives of Otorynolaryngology Head and Neck Surgery, 121:1386-1391 (1995)].

HPV infection is usually diagnosed in female patients by a combination of a visual pelvic examination, a Papanicolaou (Pap) smear (a microscopic examination of cervical cells), a colposcopy, and a DNA Pap test. In male patients, a visual exam and a DNA test are common diagnostic methods. The DNA test is most definitive.

The DNA test is broadly based on three different techniques such as non-amplification methods (e.g. Southern blotting), signal amplification tests (e.g. hybrid capture), and DNA amplification (e.g. polymerase chain reaction). Modern polymerase chain reaction is being increasingly used in clinical laboratories for the diagnosis of human papilloma virus, although amplifying HPV 53 and 61 has been problematic.

After HPV diagnosis, the treatment of HPV infection usually involves removal of any warts or abnormal tissue; however, this does not mean that the HPV infection is gone. There is high probability of regrowth and/or recurrence of the abnormal tissue.

A historical method for removing warts or other abnormal tissue was through the topical application of podophyllotoxin or trichloroacetic acid (TCA). However due to irritation and toxicity effects, neither TCA nor podophyllotoxin is now used. A different method for removing warts or other abnormal tissue is through the use of liquid nitrogen (cryotherapy). This is an unpopular method as well because of the damage to surrounding tissues during the freezing of the abnormal tissues. Another treatment for visible anogenital warts is imiquimod (Aldara®); however, the same irritation and toxicity concerns exist as for podophyllotixin and TCA (Aldara® package insert, August 2002). Also, the safety of these treatments during pregnancy has not yet been established. Other methods for wart or abnormal tissue removal include carbon dioxide treatment, laser ablation therapy, and administration of various drugs such as acyclovir derivatives, methotrexate, interferon, or retinoic acid.

There are also several surgical procedures available such as Loop Electrosurgical Excision Procedure (LEEP) and Cold Knife Cone. There are risks of infection and other complications associated with these surgical procedures. A hysterectomy can also be performed to remove abnormal tissue around and related to the cervix but there is no chance of pregnancy after this procedure and there can be hormonal and emotional complications as well. As for the development of a HPV vaccine, HPV is viewed as an ineffective agent of low immunogenicity and this, combined with the large number of genotypes, makes the development of a vaccine extremely problematic.

No presently used therapy is completely satisfactory, and, although the life expectancy of patients has been considerably increased by present therapy, the ravages of therapeutic side effects and surgery take a severe toll in well-being, general health, and increased morbidity and mortality of the millions of Americans with this disease.

It would therefore be beneficial if a non-toxic and non-irritating composition for the topical treatment of HPV infection was available. The description that follows demonstrates such a composition and method of treatment for HPV infection.

### BRIEF SUMMARY OF THE INVENTION

The present invention relates to a stable composition for use as a non-irritating and non-toxic topical treatment of prevention of human papilloma virus (HPV) infection. More particularly, the present invention contemplates a method for enhancing the chemical stability of a dietary indole or derivative thereof in a topical pharmaceutical composition and the composition itself. In a preferred embodiment, a dietary indole compound-complexing amount of hydroxypropyl-β-cyclodextrin is added to enhance stability of an anti-viral-effective amount of the dietary indole or derivative thereof in the topical pharmaceutical composition. Preferably, the dietary indole is selected from the group consisting of indole-3-carbinol (I3C), diindolylmethane (DIM), and derivatives thereof. Preferably also, the anti-viral effective amount of dietary indole or derivative thereof present in the formulation is about 0.015 to about 10 percent by weight, and more preferably about 0.5 to about 2 percent by weight, and the amount of hydroxypropyl-β-cyclodextrin present is typically about 3 to about 15 percent by weight, and more preferably about 4 to about 7 weight percent.

In another embodiment, the present invention contemplates a chemically stable composition containing an anti-viral effective amount of one or more synthetic, semi-synthetic, and naturally occurring dietary indoles and their derivatives including indole-3-carbinol (I3C), diindolylmethane (DIM) in conjunction with a dietary indole compound-complexing amount of hydroxypropyl-β-cyclodextrin for topical treatment at the site of human papilloma virus (HPV) infection. Preferably, the composition further comprises an effective amount of one or more of an antiviral agent, antiseptic agent, chemotherapeutic agent, immunopotentiating agent, or mixtures thereof.

In yet another embodiment, the present invention contemplates a method for the topical treatment or prevention of cervical dysplasia that comprises applying a composition containing an anti-viral effective amount of a dietary indole and a dietary indole compound-complexing amount of hydroxypropyl-β-cyclodextrin on the surface of the cervix.

### BRIEF DESCRIPTION OF THE DRAWINGS

In the drawings forming a part of this disclosure,

FIG. 1 is a graph depicting the stability profile of I3C over time at two different temperatures in which diamonds show data at 5° C. and the squares show data at room temperature (RT);

FIG. 2 is a graph depicting the permeation profiles for I3C through a model membrane, wherein data shown in diamonds are from Composition 3; data shown in squares are from Composition 4; and data shown in triangles are from Composition 9; and

FIG. 3 is a bar graph depicting the retention of I3C in a model membrane after 4 hours, wherein Composition 3 is about 35 percent, Composition 4 is about 40 percent and Composition 9 is less than about 5 percent, and the line above each bar depicts the standard deviation.

### DETAILED DESCRIPTION OF THE INVENTION

The effectiveness of dietary indoles, such as indole-3-carbinol (I3C), and 3,3′-diindoylmethane (DIM) 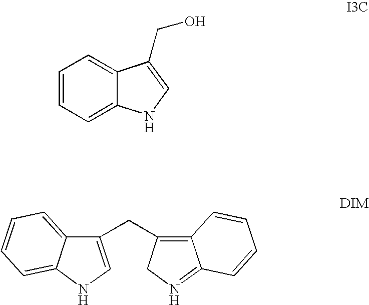
in the treatment of human papilloma virus (HPV) infections high risk types associated with HPV 16, 18, 31, 33, and 45 (cervical dysplasia, cancer of cervix) and low risk types HPV 1-5 (cutaneous warts) 2,6,11,13, and 32 (respiratory system warts) is well documented [Jin. et al., Cancer Res. 59:3391-3397 (1999); Cassie et al., Chem.-Biol. Interact. 102:1-16 (1996); Chang et al., Biochem. Pharmacol. 825-834 (1999); Yuan et al., Anticancer Research 19:1673-1680 (1999), Chinni and Sarkar, Clin. Cancer Res. 8:1228-1236 (2002)].

In addition to the effectiveness of a dietary indole on the treatment of HPV infections, various investigations have shown their effectiveness in other areas such as the ability to impede the growth of breast cancer tumors [Wattenberg and Loub, Cancer Res. 38:1410-1415 (1978); and Grubbs et al., Anticancer Res. 15:709-716 (1995)], chemoprevention and treatment of neoplasia (U.S. Pat. No. 6,399,645), adjustment of steroid hormone metabolism as anti-estrogens (U.S. Pat. No. 6,086,915), treatment of premenstrual syndromes and menopause, weight loss promoters (U.S. Pat. No. 6,534,085) and oral absorption enhancing agents (U.S. Pat. No. 6,416,793).

Although dietary indoles can be useful and effective for treating many diseases, at the present time their practical use is limited. For instance, I3C is chemically very unstable and does not completely survive exposure to gastric acid in the low-pH environment of the stomach [de Kruif et al., Chem.-Biol. Interact. 80:303-315 (1991)]. There, I3C is converted into several natural indole derivatives such as its dimer 3,3′-diindolylmethane (DIM) and indolo[3,2-b]carbazole (ICZ) through an acid-catalyzed reaction. However, these derivatives also have some biological activity although it is lower than I3C. For example, DIM has been somewhat effective in reducing DMBA-induced mammary tumors, but not consistently as effective as I3C [Wattenberg and Loub, Cancer Res. 38:1410-1415 (1978)].

To enhance the stability of I3C, Firestone et al. (U.S. Pat. No. 6,656,963) chemically synthesized derivatives of I3C, primarily esters of I3C. Their data indicate that I3C itself, and not its acid breakdown products, is a potent anti-tumor agent. However, their work also showed that stable derivatives of I3C can be used to inhibit the growth of estrogen-dependent or independent breast cancer cells and other types of cancer cells.

To overcome the existing difficulties in the pharmaceutical use of I3C, the present invention provides a method to stabilize a dietary indole in an aqueous topical pharmaceutical composition. Hydroxypropyl-β-cyclodextrin is admixed with the dietary indole in order to provide stability from degradation to the indole in the formulation, presumably by formation of an inclusion complex of one indole-containing molecule with one hydroxypropyl β-cyclodextrin molecule.

Thus, one embodiment of the invention contemplates a topical pharmaceutical composition that comprises an aqueous medium containing an anti-viral-effective amount of the dietary indole or derivative thereof and a dietary indole compound-complexing amount of a hydroxypropyl-β-cyclodextrin dissolved or dispersed therein. The hydroxypropyl-β-cyclodextrin provides enhanced stability from degradation to the dietary indole as compared to the stability of the dietary indole or its derivative in a similar composition that contains water in place of the hydroxypropyl-β-cyclodextrin after weeks of storage at a temperature of 5° C. Thus, when one assays for the presence of the dietary indole or its derivative in two compositions that differ only in the fact that one has a dietary indole compound-complexing amount of a hydroxypropyl-β-cyclodextrin and the other contains water in place of the cyclodextrin compound, one finds more dietary indole compound in the compound containing the cyclodextrin compound.

A further embodiment of the invention contemplates a topical pharmaceutical composition that comprises polyethylene glycol, a medium containing an anti-viral-effective amount of the dietary indole or derivative thereof and a dietary indole compound-complexing amount of a hydroxypropyl-β-cyclodextrin dissolved or dispersed therein. The polyethylene glycol has a molecular weight of about 200 to about 2000 and is preferably one or both of; i.e., a mixture of, PEG 400 and PEG 1450. Further the polyethylene glycol can have a weight percent in the composition of about 55 to about 70 percent and preferably from about 60 to about 65 percent.

A still further embodiment of the invention contemplates a topical pharmaceutical composition that comprises an aqueous medium containing an anti-viral-effective amount of the dietary indole or derivative thereof and about 40 to about 70 percent by weight, preferably about 50 to about 67 percent by weight, and most preferably about 53 to 65 weight percent, of the composition sorbitol dissolved or dispersed therein.

In the present invention, preferably, the dietary indole is I3C or a derivative thereof such as DIM. The dietary indole or derivative is present in the composition in an anti-viral effective amount, whereas the β-cyclodextrin is present in an indole-complexing amount, which is an amount sufficient to complex the anti-viral effective amount. In one preferred embodiment, the amount of dietary indole or derivative thereof present in the formulation is about 0.015 percent to about 10 percent by weight and the amount of hydroxypropyl-β-cyclodextrin present is about 3 percent to about 15 percent by weight. More preferably, those amounts are about 0.5 to about 2 weight percent of the dietary indole and about 4 to about 7 weight percent of the hydroxypropyl β-cyclodextrin.

In another embodiment, the present invention provides a composition comprising an anti-viral effective amount of a dietary indole or derivative thereof and a dietary indole compound-complexing amount of hydroxypropyl-β-cyclodextrin. In this embodiment, preferably the dietary indole is indole-3-carbinol (I3C), diindolylmethane (DIM), or derivatives thereof. More preferably, the amount of dietary indole present in the formulation is about 0.015 percent to about 10 percent by weight and the amount of hydroxypropyl-β-cyclodextrin present is about 3 percent to about 15 percent by weight. More preferably still, those amounts are about 0.5 to about 2 weight percent of the dietary indole and about 4 to about 7 weight percent of the hydroxypropyl β-cyclodextrin.

The composition can further comprise antiviral agents, antiseptic agents, chemotherapeutic agents, immunopotentiating agents, or mixtures thereof.

A contemplated derivative of a dietary indole can be a pharmaceutically-acceptable salt. The term “pharmaceutically-acceptable salt” encompasses those salts that form with a protonated nitrogen atom of a dietary indole and include salts formed with the organic and inorganic acids discussed below. Such acids include without limitation inorganic acids such as hydrochloric, hydrobromic, sulfuric, bisulfonic, and phosphoric, and C₁-C₂₄ organic acids such as formic acid, acetic, succinic, citric, isocitric, lactic, maleic, malic, fumaric, cholic, gluconic, glucuronic, pyruvic, oxalacetic, alginic, aspartic, glutamic, pamoic, mucic, D-glutamic, d-camphoric, glutaric, phthalic, tartaric, salicyclic, camphoric, camphorsulfonic, digluconic, methanesulfonic, benzenesulfonic, toluenesulfonic, sorbic, picric, benzoic, cinnamic, and like acids.

A contemplated derivative such as I3C that contains a hydroxyl group can also be an ester of a C₁-C₂₄ pharmaceutically acceptable acid. These acids include those enumerated above as well as straight and branched chained, saturated and unsaturated acids such as propionic, valeric, caproic, caprylic, capric, lauric, oleic, myristic, palmitic, stearic, linoleic, linolenic and eicosanoic.

A derivative of a dietary indole such as I3C that is an ester can also be a salt because the indole nitrogen atom can be protonated. Thus, a derivative can be one or both of a pharmaceutically-acceptable salt and an ester of a C₁-C₂₄ pharmaceutically acceptable acid formed from a dietary indole that contains a hydroxyl group.

In yet another embodiment, the present invention provides a method for the topical treatment or prevention of cervical dysplasia that comprises applying a composition containing an anti-viral effective amount of a dietary indole or derivative thereof and a dietary indole compound-complexing amount of hydroxypropyl-β-cyclodextrin to the surface of the cervix. Preferably, the dietary indole is indole-3-carbinol (I3C), diindolylmethane (DIM), or a derivative thereof. More preferably, the amount of dietary indole or derivative thereof present in the composition is about 0.015 percent to about 10, and more preferably about 0.5 to about 2, percent by weight and the amount of hydroxypropyl-β-cyclodextrin present is about 3 percent to about 15, more preferably about 4 to about 7, percent by weight. In this embodiment, the composition can also comprise antiviral agents, antiseptic agents, chemotherapeutic agents, immunopotentiating agents, or mixtures thereof.

In a complimentary embodiment, the present invention also provides a method for the topical treatment or prevention of human papilloma virus infection, which comprises applying a composition that contains an anti-viral effective amount of a dietary indole or derivative thereof and a dietary indole compound-complexing amount of hydroxypropyl-β-cyclodextrin at the site of infection. Preferably, the dietary indole is indole-3-carbinol (I3C), diindolylmethane (DIM), or a derivative thereof. More preferably, the amount of dietary indole present in the composition is about 0.015 to about 10 percent by weight and the amount of hydroxypropyl-β-cyclodextrin present is about 3 to about 15 percent by weight. More preferably, those amounts are about 0.5 to about 2 weight percent of the dietary indole and about 4 to about 7 weight percent of the hydroxypropyl β-cyclodextrin.

The composition can further comprise an effective amount of one or more antiviral agents, antiseptic agents, chemotherapeutic agents, immunopotentiating agents, or mixtures thereof. Preferably, the immunopotentiating agent is selected from the group consisting of acyclovir, vidarabine, uridine, 5-fluorouracil, thiotepa, interferons, podophyllotoxin, trichloroacetic acid, salicylic acid, carbamide peroxide, hexamethylene tetramine, and derivatives thereof. It has been shown that these compounds are effective in reducing abnormal skin growth conditions [Atlas, Microbiology, Fundamentals and Applications, p 551, MacMillan, New York, London, (1984)]. In addition, 5-fluorouracil and thiotepa exhibit significant antineoplastic activities. The usage of podophyllotoxin, trichloroacetic acid, and salicylic acid is also documented in the treatment of HPV infections and warts.

It is also known that interferons, which are released from infected cells, migrate to uninfected cells and protect them from viral infections [Atlas, Microbiology, Fundamentals and Applications, p. 481, MacMillan, New York, London, (1984)]. The activity of interferon on the cell lines of human papilloma virus (especially type 31) as manifested by growth arrest and apoptosis is also reported [Chang et al., J. Virol. 76:8864-8874 (2002)]. Other antimicrobial, antiseptic and or oxygen donating agents, such as carbamide peroxide [Lim et al., Cytotechnology 31:265-270 (1999)] or other germ killing compound generating agents such hexamethylene tetramine can also help to eradicate viruses.

In another embodiment, the composition can further comprise a pharmaceutically acceptable base. Illustrative pharmaceutically acceptable bases include basic amino acids selected from the group consisting of arginine, lysine, proline, as well as an amine selected from the group consisting of diethanolamine, triethanolamine (Trolamine), trimethylamine, diethylamine, procaine, N,N′-dibenzylethylenediamine, hexamethylenetetramine chloroprocaine, choline, ethylenediamine, and meglumine (N-methylglucamine). Mixtures of pharmaceutically acceptable bases are also contemplated. These bases are used to adjust the pH value of the composition from about 7.0 to about 9.0, and preferably about 7 to about 8.

A contemplated composition is designed to be used topically. As such, it is preferred that the viscosity of the composition be greater than that of drinking water. It is preferred that the viscosity be between that exhibited by heavy cream or motor oil and mayonnaise at room temperature. Thus, one preferred composition can be poured readily at room temperature. Other preferred compositions tend to pour only with great difficulty if at all at room temperature, and maintain a peaked shape for several seconds after being scooped from a container and formed in to a mound in another.

These ranges in viscosity are typically achieved with the assistance of one or more polymeric thickeners. Illustrative polymeric thickeners include the starch derivatives Zeina B862 hydroxypropyl starch phosphate, and Zeina B860 hydroxypropyl starch. A polysaccharide gum is another useful thickener that can be present in a contemplated composition. Suitable representative gums are those in the galactomannan gum category. A galactomannan gum is a carbohydrate polymer containing D-galactose and D-mannose units, or other derivatives of such a polymer. There is a relatively large number of galactomannans, which vary in composition depending on their origin. The galactomannan gum is characterized by a linear structure of β-D-mannopyranosyl units linked (1→6). Single membered α-D-manopyranosyl units, linked (1→6) with the main chain, are present as side branches. Galactomannan gums include guar gum, which is the pulverized endosperm of the seed of either of two leguminous plants (Cyamposis tetragonalobus and psoraloids) and locust bean gum, which is found in the endosperm of the seeds of the carob tree (Ceratonia siliqua). Locust bean gum is preferred for the present invention.

Other suitable representative gums include agar gum, hydroxypropyl guar gum (Jaguar HP 120), carrageenan gum, ghatti gum, karaya gum, rhamsan gum and xanthan gum. A composition of the present invention can contain a mixture of various gums, or mixture of gums and acidic polymers.

Gums, and galactomannan gums in particular, are well-known materials. See for instance, Industrial Gums: Polysaccharides & Their Derivatives, Whistler R. L. and BeMiller J. N. (eds.), 3rd Ed. Academic Press (1992) and Davidson R. L., Handbook of Water-Soluble Gums & Resins, McGraw-Hill, Inc., New York (1980). Most gums are commercially available in various forms, commonly a powder, and ready for use in foods and topical compositions. For example, locust bean gum in powdered form is available from Tic Gums Inc. (Belcam, Md.).

A polysaccharide gum, when used, is present at about 0.5 percent to about 5 percent, based on the total weight of the composition, with the preferred amount being about 0.5 percent to about 2 percent.

An alternative or addition to the polysaccharide gum is a polyacrylic acid polymer. A common variety of polyacrylic acid polymer is known generically as “Carbomer” that are polyacrylic acid polymers lightly cross-linked with polyalkenyl polyether. There materials are commercially available from the B. F. Goodrich Company (Akron, Ohio) under the designation “CARBOPOL®”. A particularly preferred variety of carbomer are those designated as “CARBOPOL 940” and “CARBOPOL 934”.

Other polyacrylic acid polymers suitable for use in practicing this invention are those commercially available under the designations “Pemulen®” (B. F. Goodrich Company) and “POLYCARBOPHIL®” (A. H. Robbins, Richmond, Va.). The Pemulen® polymers are copolymers of C₁₀ to C₃₀ alkyl acrylates and one or more monomers of acrylic acid, methacrylic acid or one of their simple esters cross-linked with an allyl ether of sucrose or an allyl ether of pentaerythritol. POLYCARBOPHIL® is a polyacrylic acid cross-linked with divinyl glycol. POLYCARBOPHIL® is used in the vaginal moisturizer disclosed in U.S. Pat. No. 5,474,768.

The levels and incorporation of polymeric thickeners into the formulations are based on the well-established pharmaceutical principles. Other formulation components such as coloring agents, antioxidants, emollients, glidants, and antimicrobials can also be added.

It is also to be understood that in therapeutic formulations the active materials can be dispersed, dissolved, emulsified, suspended, lyophilized, encapsulated, micronized, nanoparticulated and the like. In addition, the formulations can contain solvents such as alcohols, polyols, esters, water; thickening and consistency modulating agents such as galactomannan gums, carbohydrate polymers such as starch, cellulose derivatives, alginic acid and derivatives, alkyl acrylate polymers and copolymers, polyvinyl alcohol and derivatives; film forming agents such as cellulose ethers, carbomers; chelating agents, emulsifying agents such as long chain fatty acids, regular or fatty alcohols, and or esters; amino acids, and buffering agents.

The addition of film forming agents facilitates the retention and the adhesion of the formulation to the infection area so that the drug is delivered directly at the site of infection. The utilization of devices especially designed to deliver the formulations to the exact location which are difficult to reach, such as cervix, is within the scope of this invention.

The formulations containing I3C, DIM, and derivatives thereof can be prepared by stepwise dissolving, dispersing, and emulsifying the active agent in the appropriate phase, followed by the successive addition of thickening agents, chelating agents, formulation aids, and other ingredients. If needed, the pH can be adjusted.

Another embodiment of the present invention provides a composition comprising about 0.015 percent to about 10 percent of a dietary indole, about 5 percent to about 95 percent of a solvent, zero to about 20 percent of a thickening agent, zero to about 10 percent of a film forming agent, a buffering agent to adjust pH value, a basic amino acid or a basifying agent to adjust pH value, about 3 to about 15 percent of a chelating agent, an antimicrobial agent, and an antioxidant. More preferably, the amounts of the dietary indole and the hydroxypropyl β-cyclodextrin are as noted before.

Alternatively antiviral, antiseptic, chemotherapeutic, and immunopotentiating agents can be added to the formulations that comprise I3C or DIM, an aqueous polyol, an alcohol, a polymeric thickener, an adhesive film forming agent, a basic amino acid and buffering reagents, a chelating agent and other pharmaceutical aids.

In the present invention, the formulations were filled into various plastic (HDPE) applicators consisting of a barrel, a piston, a specially designed tip, and a cap, and or various disposable polyethylene syringes (1 to 2 mL) fitted with an extended tip. The size of the applicators can be designed for specific body locations and/or to contain premeasured amounts of the formulation to be delivered. Applicators can be manufactured from chemically or surface treated polyethylene or other medicinally acceptable polymers such as polypropylene, fluorinated polymers or similar materials. Also the formulations can be embedded within the plastic or metal delivery devices.

An additional embodiment comprises a method for treating or preventing abnormal cell growth of I3C and DIM which comprises applying a composition comprising about 0.015 percent to about 10 percent I3C and DIM, a solvent, a polymer, an adhesive film forming agent, a basic amino acid or buffering agent, and a chelating agent. The previously noted preferred amounts of dietary indole and cyclodextrin compound are useful here also.

Preferably, the composition is provided for use within a delivery device (applicator or dispenser). More preferably, the delivery device is made from a material that is selected from the group consisting of chemically or surface treated polyethylene, polypropylene, glass, metal, and fluorinated polymers.

In a separate embodiment, the present invention provides a method for enhancing the chemical stability of a dietary indole or derivative thereof in a pharmaceutical formulation that comprises adding a chelating agent to the formulation.

In a different embodiment, the present invention contemplates a method for the topical treatment or prevention of abnormal cell growth that comprises applying a composition containing an anti-viral effective amount of a dietary indole or derivative thereof and a dietary indole compound-complexing amount of hydroxypropyl-β-cyclodextrin on to the surface of the abnormal cells. The abnormal cell growth contemplated for treatment is a condition that is a member of the group consisting of a palmar wart, a plantar wart, a cutaneous wart, a childhood wart, condyloma acuminatum, a genital wart located at peri-anal epithelium, anal human papilloma virus related papilloma, vaginal epithelial dysplasia, cervical dysplasia, oral infections such as recurrent laryngeal papillomatosis, oropharyngeal human papilloma virus related papilloma and dysplasia, tracheal infections, nasopharyngeal infections, and the undersurface of the vocal folds, carina, and bronchial spurs. The present invention provides a method for the prevention of abnormal cell growth that comprises applying a composition that contains an anti-viral effective amount of a dietary indole or derivative thereof and a dietary indole compound-complexing amount of hydroxypropyl-β-cyclodextrin on the surface of the abnormal cells.

The application of I3C, DIM, and like can be facilitated using various dosage forms including solid dosage forms, creams, ointments, suspensions, liniments, solutions, sprays, aerosols, metered dose inhalers, nebulizers, powders, pessaries, transdermal patches, iontophoretic, phonophoretic delivery systems, monolithic devices and other pharmaceutically acceptable forms.

The present invention is further described by following examples.

| | COMPOSITION AND EXAMPLE NUMBER | | | | | | | |
|---|---|---|---|---|---|---|---|---|
| | 1 | 2 | 3 | 4 | 5 | 6 | 7 | 8 |
| INGREDIENT | WEIGHT PERCENT OF TOTAL COMPOSITION | | | | | | | |
| I3C | 1 | 1 | 1 | 1 | 1 | 1 | 1 | 1 |
| Hydroxy- | 2.5 | 2.5 | 2.5 | 2.5 | 2.5 | 2.5 | 2.5 | 2.5 |
| propyl Starch | | | | | | | | |
| Phosphate | | | | | | | | |
| Ethanol | 5 | 5 | 5 | 5 | 5 | 5 | 5 | 5 |
| Propylene | — | — | — | — | 5 | 5 | — | — |
| Glycol | | | | | | | | |
| Isopropyl | 3 | 3 | 3 | 3 | 3 | 3 | 3 | 3 |
| Myristate USP | | | | | | | | |
| Injectable | 87.5 | 82.5 | 82.5 | 87.5 | 77.5 | 82.5 | 82.5 | 87.5 |
| water USP pH | | | | | | | | |
| about 7.4 with | | | | | | | | |
| Trolamine | | | | | | | | |
| Polycarbophil | 1 | 1 | 1 | 1 | 1 | 1 | 1 | 1 |
| USP | | | | | | | | |
| Hydroxy- | — | 5 | 5 | — | 5 | — | 5 | — |
| propyl- | | | | | | | | |
| β-cyclodextrin | | | | | | | | |

### Preparation of Compositions 1-8

The desired amount of I3C was dissolved under constant stirring in a mixture of ethanol (200 proof USP) isopropyl myristate USP and/or propylene glycol in a stainless steel container. An appropriate amount of hydroxypropyl-β-cyclodextrin was also added. In a second stainless steel container, the pH value of an appropriate amount of water was adjusted to about 8.5 using Trolamine or lysine, followed by the addition of modified food starch and Polycarbophil USP under rigorous and continuous mixing for at least 1 hour until homogeneous.

The contents of the first stainless steel container were added to the second container under continuous mixing. The mixture was then stirred for about 60 minutes.

All formulations were protected from light and excessive temperatures during the preparation.

| | COMPOSITION AND EXAMPLE NUMBER | | | | | |
|---|---|---|---|---|---|---|
| | 9 | 10 | 11 | 12 | 13 | 14 |
| INGREDIENT | WEIGHT PERCENTAGE OF TOTAL | | | | | |
| I3C | 2 | 2 | 2 | 2 | 2 | 2 |
| Ethanol, 200 proof, | 5 | 10 | 5 | 10 | 5 | 10 |
| USP | | | | | | |
| Propylene glycol, NF | 5 | — | 5 | — | 5 | — |
| Hydroxypropyl-β- | — | — | 5 | 5 | 5 | 5 |
| cyclodextrin | | | | | | |
| PEG 400, NF | 65 | 65 | 60 | 60 | 60 | 60 |
| PEG 1450, NF | 23 | 23 | 23 | 23 | 22.5 | 22.5 |
| Trolamine | — | — | — | — | 0.5 | 0.5 |

### Preparation of Compositions 9-14

The desired amount of I3C was dissolved under constant stirring in a mixture of ethanol (200 proof USP) isopropyl myristate USP and/or propylene glycol in a stainless steel container. An appropriate amount of hydroxypropyl-β-cyclodextrin was also added and the pH was adjusted to about 8.5 using Trolamine, followed by the addition of modified food starch and Polycarbophil USP under rigorous and continuous mixing for at least 1 hour until homogeneous.

In a second stainless steel container PEG 400 and PEG 1450 were mixed and dissolved under gentle heating and kept at 37° C. The contents of the second container were carefully and quantitatively transferred in to the first container under constant stirring and cooling. The contents were mixed for additional 60 minutes.

All formulations were protected from light and excessive temperatures during the preparation.

| | COMPOSITION AND EXAMPLE NUMBER | |
|---|---|---|
| | 15 | 16 |
| INGREDIENT | WEIGHT PERCENTAGE OF TOTAL | |
| I3C | 1 | 1 |
| Sorbitol 70% USP | 75.5 | 80.5 |
| Diethylene Glycol | 10 | 10 |
| Monoethyl Ether NF | | |
| Hydroxypropyl Starch | 6.5 | 6.5 |
| Phosphate | | |
| Polycarbophil USP | 1 | 1 |
| Lysine USP | 1 | 1 |
| Hydroxypropyl-β- | 5 | — |
| cyclodextrin | | |

### Peparation of Compositions 15-16

The desired amount of I3C was dissolved and dispersed under constant stirring in diethylene glycol monoethyl ether NF. An appropriate amount of hydroxypropyl-β-cyclodextrin was then added.

In a second stainless steel container lysine was dispersed in sorbitol 70 percent USP. This was followed by the addition of modified food starch and polycarbophil under rigorous and continuous mixing for at least 1 hour. The resulting mixture was stirred until homogeneous.

The contents of the first stainless steel container were added to the second container under continuous mixing. After the completion of the addition the stirring continued for additional 1 hour. All process above was performed under protection from the light. After filling the containers were blanketed with nitrogen, wrapped in aluminum foil, and kept in a refrigerator until testing and assay.

### Stability of the Formulations

Stability testing of the formulations was performed by HPLC analysis. The area of the peak representing I3C or a derivative thereof was monitored. The amount of the active material was determined using a calibration curve constructed by using the pure material as reference standard.

The formulations remained colorless during the stability study period without any formation of particles and no change of consistency was apparent.

The stability of I3C in aqueous media appeared to be influenced by pH value. In aqueous medium at pH 6.0 after 2 weeks at 5° C. the active was completely decomposed whereas at pH 8.0 the amount of the active was at about 93% level (see Table 1, below). However, in combination with additional ingredients such as chelating agents and cyclodextrins, long-term stability of formulations was achieved. For example, I3C in solution at pH 7.4 with added hydroxypropyl-β-cyclodextrin loses 1.3 percent of its potency after 2 weeks, whereas with the same aqueous composition without hydroxypropyl-β-cyclodextrin I3C loses 16 percent of its potency after 2 weeks.

Because cyclodextrins are available at different substitution ranges hydroxypropyl-β-cyclodextrin at 0.6, 0.8, and 1.0 substitution degrees were evaluated. The stability studies showed no significant effect of the substitution on the decomposition rates of the products.

TABLE 1
Stability results at a temperature of 5° C.

| | | Hydroxypropyl-β- | | |
|---|---|---|---|---|
| | pH | cyclodextrin | Time | Percent |
| Type | Value | (1.0%) | (Days) | I3C Left |
| Aqueous solution | 6.0 | No | 15 | 0 |
| (Control) | | | | |
| Aqueous solution | 7.0 | No | 15 | 72 |
| (Control) | | | | |
| Aqueous solution | 7.4 | No | 15 | 84 |
| (Control) | | | | |
| Aqueous solution | 7.4 | Yes | 15 | 98.7 |
| (Control) | | | | |
| Aqueous solution | 8.0 | No | 15 | 93 |
| (Control) | | | | |
| Composition 1 | 7.4 | No | 30 | 92 |
| Composition 2 | 7.4 | Yes | 30 | 98.5 |

When evaluating the stability of the composition adjusted to pH 7.4 without hydroxypropyl-β-cyclodextrin (1.0%) after one month, 92 percent active was present, whereas the incorporation of hydroxypropyl-β-cyclodextrin (1.0%) increased the level of I3C to 98.5 percent.

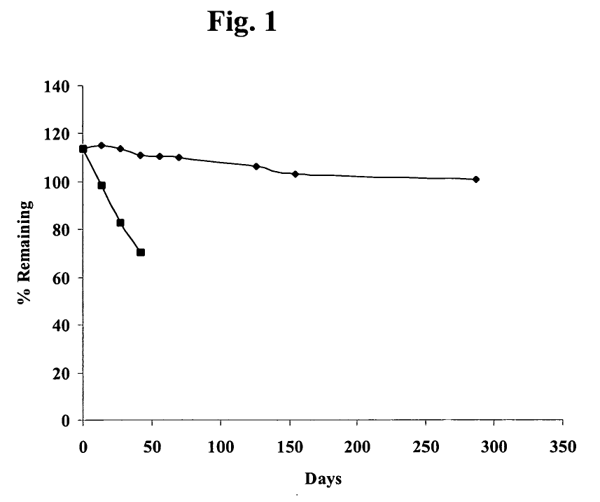

The stability of Composition 3 was followed at refrigerated temperature (2° C. to 8° C.) during a period of 10 months (FIG. 1). As shown from the graph, at the end of 10 months of storage, over 95 percent active material remained available at refrigerated temperature whereas at room temperature (RT) conditions, I3C decomposed very rapidly. Therefore, pH adjustment in combination with the addition of cyclodextrin improves the stability of I3C significantly.

From the data it is apparent that Composition 3 exhibits good I3C stability. The stabilization of I3C in solution or semisolid formulations has not been described in the art and is completely new.

Because aqueous formulations of I3C are subject to decomposition and are strongly dependent on pH value, additional non-aqueous formulations were also developed. The formulations are described as examples 9-14. Typical stability studies showed that they exhibited similar shelf life characteristics of aqueous formulations.

### Skin Permeation Behavior of Some Typical Formulations

The utility of the shed snake skin model and the details of the experimentation have been demonstrated in the literature [Buyuktimkin et al. Pharm. Res. (1993)]. Briefly, previously prepared pieces of shed skin from a black rat snake (Elaphe obsoleta) were cut in pieces of about 3×3 cm. The receptor compartments of modified Franz cells were filled with pH 7.4, 0.1 M phosphate buffer and after the introduction of a small magnet they were covered with snake skin pieces. Donor and receptor compartments of the modified Franz cells were clamped together. During this process extra care was taken to remove any air bubbles. The compositions of the present invention were applied to the top of skin of the respective cells. After loosely covering the modified Franz cells with paraffin film, they were placed into thermostated water bath at 37° C. equipped with magnetic stirrers at 300 rpm. At predetermined time intervals, samples were taken then assayed by HPLC.

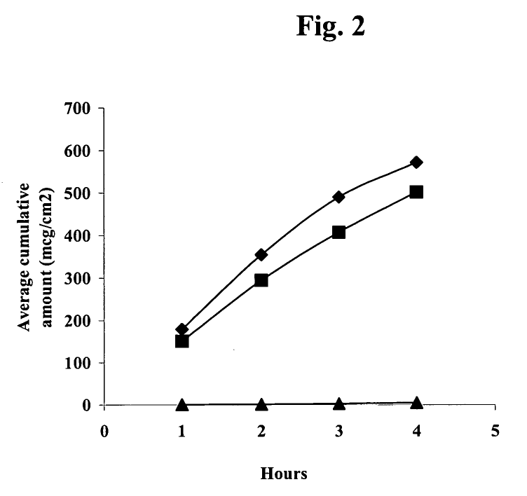

The skin permeation behavior of a typical formulation (Example 3) compared to a control formulation (Example 4) was examined using shed snakeskin as the model in vitro membrane (FIG. 2).

### Membrane Retention Behavior of Typical Formulations

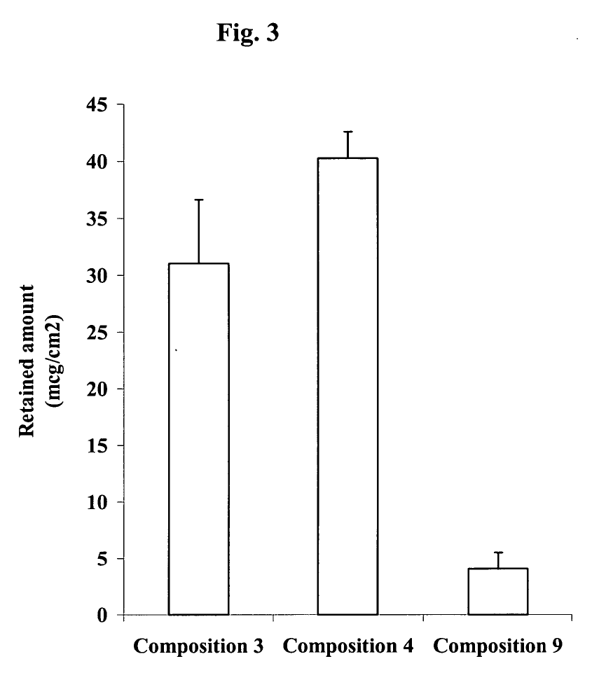

In addition to the permeation, the retention of the drug in the cells was also examined. For this purpose, the skins were carefully removed. After wiping the excess of formulation the skins were quickly washed with small amount of water (about 1 mL) and quickly tapped with a lint free paper. The skins were cut into pieces and homogenized with a hand held homogenizer in 2 mL absolute ethanol. After transferring 1 mL of liquid phase into a 10 mL volumetric flask, they were filled to the volume with the mobile phase utilized in stability studies. The contents were assayed by HPLC as described above. The retention behaviors of examples after 4 hours of study are illustrated in FIG. 3.

Each of the patents and articles cited herein is hereby incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

1. A topical pharmaceutical composition that comprises an aqueous medium containing an anti-viral-effective amount of the dietary indole or derivative thereof and a dietary indole compound-complexing amount of a hydroxypropyl-β-cyclodextrin dissolved or dispersed therein, said hydroxypropyl-β-cyclodextrin providing enhanced stability from degradation to the dietary indole as compared to the stability of said dietary indole in a similar composition that contains water in place of said hydroxypropyl-β-cyclodextrin after weeks of storage at a temperature of 5° C.

2. The topical pharmaceutical composition according to claim 1 wherein said composition has a pH value of about 7.0 to about 9.0.

3. The topical pharmaceutical composition according to claim 1 wherein said dietary indole is indole-3-carbinol or 3,3′-diindoylmethane.

4. The topical pharmaceutical composition according to claim 1 wherein the dietary indole derivative is one or both of a pharmaceutically-acceptable salt and an ester of a C₁-C₂₄ pharmaceutically acceptable acid formed from a dietary indole that contains a hydroxyl group.

5. The topical pharmaceutical composition according to claim 1 wherein said dietary indole or its derivative is present in the composition at about 0.015 to about 10 percent by weight.

6. The topical pharmaceutical composition according to claim 1 wherein said hydroxypropyl-β-cyclodextrin is present at about 3 to about 15 percent by weight.

7. A topical pharmaceutical composition that comprises an aqueous medium containing an anti-viral-effective amount of indole-3-carbinol (I3C) or 3,3′-diindoylmethane or derivative thereof that is one or both of a pharmaceutically-acceptable salt and an ester of a C₁-C₂₄ pharmaceutically acceptable acid formed from I3C and a dietary indole compound-complexing amount of a hydroxypropyl-β-cyclodextrin dissolved or dispersed therein, said composition having a pH value of about 7.0 to about 9.0, said hydroxypropyl-β-cyclodextrin providing enhanced stability from degradation to the dietary indole as compared to the stability of said dietary indole in a similar composition that contains water in place of said hydroxypropyl-β-cyclodextrin after weeks of storage at a temperature of 5° C.

8. The composition according to claim 7 wherein said composition further contains effective amount of one or more antiviral agents, antiseptic agents, chemotherapeutic agents, immunopotentiating agents, or mixtures thereof.

9. The composition according to claim 7 wherein said composition further contains a pharmaceutically acceptable base.

10. The composition of claim 1 further for use in the topical treatment or prevention of cervical dysplasia.

11. The composition of claim 3 further for use in the topical treatment or prevention of cervical dysplasia.

12. The composition to the fed to of claim 4 further for use in the topical treatment or prevention of cervical dysplasia.

13. The composition of claim 7 further for use in the topical treatment or prevention of cervical dysplasia.

14. The composition of claim 1 further for use in the topical treatment or prevention of human papilloma virus prevention.

15. The composition of claim 3 further for use in the topical treatment or prevention of human papilloma virus prevention.

16. The composition of claim 4 further for use in the topical treatment or prevention of human papilloma virus prevention.

17. The composition of claim 7 further for use in the topical treatment or prevention of human papilloma virus prevention.

18. The composition of claim 8 where the immunopotentiating agent is selected from the group consisting of acyclovir, vidarabine, uridine, 5-fluorouracil, thiotepa, interferons, podophyllotoxin, trichloroacetic acid, salicylic acid, carbamide peroxide, hexamethylene tetramine, and derivatives thereof.

19. The composition of claim 9 where the pharmaceutically-acceptable bases include basic amino acids selected from the group consisting of arginine, lysine, proline, and an amine selected from the group consisting of diethanolamine, triethanolamine (Trolamine), trimethylamine, diethylamine, procaine, N,N′-dibenzylethylenediamine, hexamethylenetetramine chloroprocaine, choline, ethylenediamine, and meglumine (N-methylglucamine).

20. The composition of claim 1 further including a chelating agent.

21. A topical pharmaceutical composition that comprises an medium containing a polyethylene glycol, an anti-viral-effective amount of the dietary indole or derivative thereof and a dietary indole compound-complexing amount of a hydroxypropyl-β-cyclodextrin dissolved or dispersed therein, said hydroxypropyl-β-cyclodextrin providing enhanced stability from degradation to the dietary indole as compared to the stability of said dietary indole in a similar composition that contains water in place of said hydroxypropyl-β-cyclodextrin after weeks of storage at a temperature of 5° C.

22. The composition of claim 21 wherein the polyethylene glycol is present in an amount of about 55 to about 70 weight percent.

23. The composition of claim 21 wherein the polyethylene glycol has a molecular weight of about 200 to about 2000.

24. The composition of claim 21 where the polyethylene glycol is PEG 400 or PEG 1450, or a mixture thereof.

25. A topical pharmaceutical composition that comprises an aqueous medium containing an anti-viral-effective amount of the dietary indole or derivative thereof and sorbitol dissolved or dispersed therein, said sorbitol providing enhanced stability from degradation to the dietary indole as compared to the stability of said dietary indole in a similar composition that contains water in place of said sorbitol after weeks of storage at a temperature of 5° C.

26. The composition of claim 25 where the sorbitol is present in an amount of about 40 to about 70 percent.
